(19) United States
(12) Patent Application Publication
(10) Pub. No.: US 2010/0035234 A1
(43) Pub. Date: Feb. 11, 2010

(54) VACCINE ASSAYS

(75) Inventors: John Donnelly, Siena (IT); Ping Wu, Danville, CA (US); George Santos, Wayland, MA (US); Marzia Monica Giuliani, Siena (IT); William Andrews, Emeryville, CA (US); Jie Chen, Pleasanton, CA (US)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: Novartis AG, Basel (CH)

(21) Appl. No.: 12/468,779

(22) Filed: May 19, 2009

Related U.S. Application Data
(60) Provisional application No. 61/054,439, filed on May 19, 2008.

(51) Int. Cl. C12Q 1/70; G01N 33/569; G01N 33/574; G01N 33/571

(52) U.S. Cl. 435/5; 435/7.2; 435/7.32; 435/7.31; 435/7.22; 435/7.23; 435/7.36; 435/7.33; 435/7.34; 435/7.37

(57) ABSTRACT

The present invention is directed to methods, assays and compositions for implementing such methods and assays for assessing efficacy of individual components in multi-component vaccines and for assessing efficacy of a vaccine against a pathogen. In one aspect, the method of assessing efficacy of a vaccine against a pathogen is a quick assay that tests for an activity correlated with efficacy such as binding in an ELISA rather than requiring the time and expense of an assay that detects actual bactericidal activity. In another aspect, the method for testing the efficacy of an individual component in a multi-component vaccine includes obtaining an immune sample from a subject inoculated with the multi-component vaccine; blocking the portion of the immune sample that recognizes the individual component such as by addition of the individual component, and testing the efficacy of the immune sample to respond to the pathogen.

### CROSS-REFERENCE TO RELATED APPLICATION

This application claims priority of an earlier filed provisional application U.S. Ser. No. 61/054,439, titled VACCINE ASSAYS, filed May 19, 2008, all of which is incorporated herein by reference in its entireties for all purposes.

### FIELD OF THE INVENTION

The present invention relates to the field of assessing vaccine efficacy. In one aspect, the present invention relates to assessing efficacy of a vaccine against a pathogen of interest by demonstration of that serum from a subject inoculated with the vaccine or a component or epitope within the vaccines includes antibodies that bind at least a component of the pathogen without demonstration of a functional response against the pathogen such as production of bactericidal antibodies.

### BACKGROUND

The ability to assess the efficacy of a vaccine is important for many aspects of both research and development of vaccines as well as use of vaccines after development has been completed.

Presently, many vaccines and vaccine candidates are tested for efficacy by a functional assay that demonstrates the ability of a serum response in a vaccinated subject to effect killing of the pathogen vaccinated against. Functional assays such as a serum bactericidal assay are used as a proxy for efficacy based upon the assumption that if the subject has produced bactericidal antibodies against the pathogen above a specified level, then the subject is protected against infection by the organism and therefore that the vaccine may be used to protect others against the pathogen. These responses are measured in mice and are a standard indicator of vaccine efficacy (e.g. see end-note 14 of reference (R5) below). Serum bactericidal activity measures bacterial killing mediated by complement, and can be assayed using human or baby rabbit complement. WHO standards require a vaccine to induce at least a 4 fold rise in SBA in more than 90% of recipients when rabbit complement is used. Published studies (Goldschneider et al.) assigned an SBA titer of 1:4 using human complement as a correlate of protection against meningococcal disease. The functional assay and threshold as used today is typically an underestimate of a vaccine's efficacy, but such underestimate is deemed in the best interest of the public. The use of such functional assays, however, are not quick as the assays often take two or more days, are not simple to use as they require a laboratory setting where the pathogen may be cultured, and are often not cost effective owing to the time and necessary equipment for performing the assays.

For any new assay used to determine whether a vaccine is likely to be efficacious against a particular strain or variant of a pathogen whether during development or after a product is on the market, one of skill in the art will want to know whether the vaccine will produce a response in the subject that will be efficacious against the particular strain or variant. Thus, there is a need for an assay that can assess whether a vaccine is efficacious against a pathogen of interest quickly and preferable economically and without requiring a fully equipped laboratory.

### SUMMARY

The present invention addresses these long felt needs by providing methods of assessing efficacy of a vaccine on a pathogen-by-pathogen basis as well as compositions for performing such methods. One aspect of the invention is based upon the surprising discovery that assays that merely detect the presence of antibodies to a vaccine component or an epitope therein such as ELISA without actually determining whether such antibodies provide a functional response against the pathogen such as bactericidal antibodies correlate sufficiently to such assays that they may be used in lieu of such assays.

One aspect of the invention is a method of assessing efficacy of a vaccine component against a pathogen wherein a pathogen sample is provided; the pathogen sample is contacted with a component binding antibody preparation; the efficacy of the vaccine component is assessed by detecting whether the component-directed antibody preparation binds to the pathogen sample. In certain embodiments, the pathogen sample used in the method is an intact pathogen cell or virus or a detergent solubilized portion of the pathogen. In some embodiments the detergent solubilized portion of the pathogen is a membrane associated protein. In some embodiments which may be combined with any of the preceding embodiments using a detergent, the detergent is a non-ionic detergent, a cationic detergent, an anionic detergent, or a zwittergent.

In certain embodiments which may be combined with any of the preceding embodiments, the detecting is performed with an ELISA assay. In some embodiments, the enzyme of the ELISA assay is selected from horse-radish peroxidase, alkaline phosphatase, β galactosidase, luciferase, and acetylcholinesterase. In some embodiments which may be combined with any of the preceding embodiments using an enzyme in the detection, a chromogenic, radiolabeled or a fluorescent substrate is used.

In certain embodiments which may be combined with any of the preceding embodiments, the pathogen is a bacterial pathogen, a viral pathogen, a fungal pathogen, or a parasite pathogen. In certain embodiments which may be combined with any of the preceding embodiments, the pathogen is N. meningitidis, N. gonorrhoeae, Streptococcus pyogenes, Streptococcus agalactiae, Streptococcus pneumoniae, H. influenzae, Staphylococcus aureus, Haemophilus influenza B, H. pylori, meningitis/sepsis associated E. coli, or uropathogenic E. coli. In certain embodiments which may be combined with any of the preceding embodiments, the pathogen is influenza, RSV, HCV, HSV, HIV-1, or HIV-2.

In some embodiments which may be combined with any of the preceding embodiments, the vaccine component is a protein, a proteoglycan, a lipoprotein, a polysaccharide, a lipopolysaccharide, a viral envelope protein in monomeric or multimeric form, an outer membrane vesicle, a virus-like particle, or an entire vaccine.

In some embodiments which may be combined with any of the preceding embodiments, the antibody preparation is a polyclonal antibody containing serum sample, polyclonal antibodies, antigen-purified polyclonal antibodies monoclonal antibodies, or a combination of two or more of the foregoing. In some embodiments which may be combined with any of the preceding embodiments, the polyclonal antibodies are directed to the vaccine, to all components of the vaccine, or to a single component of the vaccine. In certain embodiments which may be combined with any of the preceding embodiments, wherein the antibodies bind to the vaccine, to a single component of the vaccine or to an epitope of the vaccine.

In some embodiments which may be combined with any of the preceding embodiments, the pathogen is N. meningitidis serogroup B. In some embodiments which may be combined with any of the preceding embodiments, the vaccine component comprises one or more of a GNA1870 antigen, a GNA2132 antigen, and a NadA antigen.

Another aspect of the invention is a method of assessing efficacy of a vaccine multicomponent N. meningitidis serogroup B against an N. meningitidis serogroup B wherein a detergent extracted sample of the N. meningitidis serogroup B strain is provided; individual portions of the detergent extracted sample are separately contacted with a GNA1870 antigen-binding antibody preparation, a GNA2132 antigen-binding antibody preparation, and a NadA antigen-binding antibody preparation; and the efficacy of the vaccine component is assessed by detecting whether each antibody preparation binds to contacted individual portion of the detergent extracted sample. In some embodiments, the detergent is a non-ionic detergent, a cationic detergent, an anionic detergent, or a zwittergent.

In some embodiments which may be combined with the preceding embodiment, the kit includes an enzyme for detection of binding. In certain embodiments, the enzyme is a horse-radish peroxidase, alkaline phosphatase, β galactosidase, luciferase, and acetylcholinesterase. In some embodiments which may be combined with any of the preceding embodiments using an enzyme in the detection, the kit includes a chromogenic, radiolabeled or a fluorescent substrate.

In some embodiments which may be combined with any of the preceding embodiments, the antibody preparations are polyclonal antibody containing serum samples, polyclonal antibodies, antigen-purified polyclonal antibodies monoclonal antibodies, or a combination of two or more of the foregoing. In certain embodiments which may be combined with any of the preceding embodiments, the antibodies bind to the vaccine, to a single component of the vaccine or to an epitope of the vaccine.

Another aspect of the invention is a kit for practicing any of the preceding aspects or embodiments. In one embodiment, a kit will include at least one of a GNA1870 antigen-binding antibody preparation, a GNA2132 antigen-binding antibody preparation, and a NadA antigen-binding antibody preparation. In certain embodiments the kit will have all three antibody preparations. In certain embodiments which may be combined with any of the preceding embodiments, the kit will also include a detergent for extraction of a portion of a pathogen. In some embodiments, the detergent is a non-ionic detergent, a cationic detergent, an anionic detergent, or a zwittergent.

In certain embodiments which may be combined with any of the preceding embodiments, the detecting is performed with an ELISA assay. In some embodiments, the enzyme of the ELISA assay is selected from horse-radish peroxidase, alkaline phosphatase, β galactosidase, luciferase, and acetylcholinesterase. In some embodiments which may be combined with any of the preceding embodiments using an enzyme in the detection, a chromogenic, radiolabeled or a fluorescent substrate is used.

In some embodiments which may be combined with any of the preceding embodiments, the antibody preparation is a polyclonal antibody containing serum sample, polyclonal antibodies, antigen-purified polyclonal antibodies monoclonal antibodies, or a combination of two or more of the foregoing. In some embodiments which may be combined with any of the preceding embodiments, the polyclonal antibodies are directed to the vaccine, to all components of the vaccine, or to a single component of the vaccine. In certain embodiments which may be combined with any of the preceding embodiments, wherein the antibodies bind to the vaccine, to a single component of the vaccine or to an epitope of the vaccine.

Additional aspects of the invention may be found throughout the specification.

### SUMMARY OF THE FIGURES

FIG. 1 shows the correlation between the whole cell ELISA (WCE) assay showing detection of the NadA (TIGR-961 or NMB1994) on the surface of various N. meningitidis serogroup B strains (H44/76, NMB, 5/99, M4007, NZ98/254, 2996 MC58, M4458, GB364, 95N477, M1390, GB013, and M3812) and the corresponding serum bactericidal assay (SBA) from sera before and after immunization with NadA.

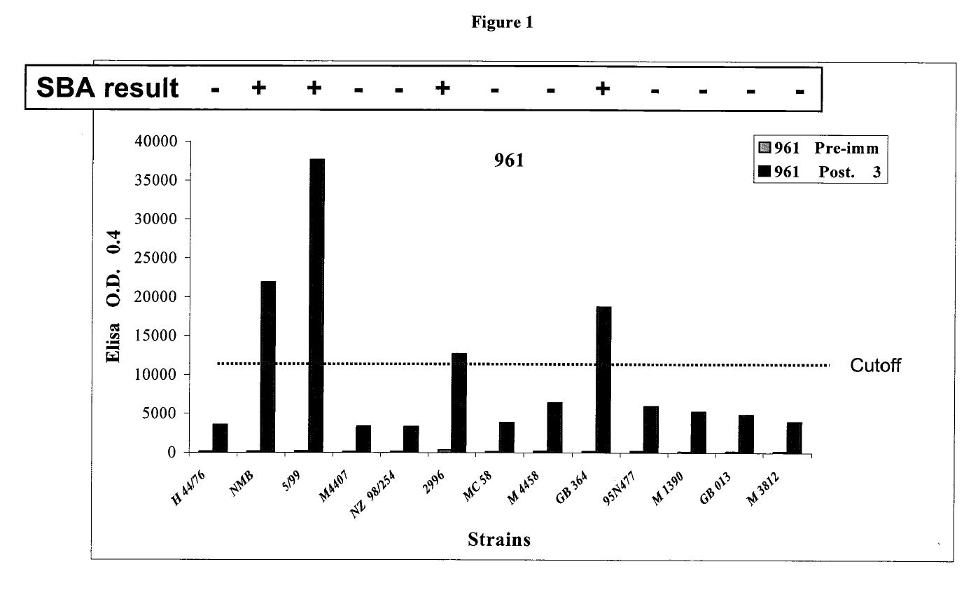

FIG. 2 shows a simpler ELISA assay than the WCE where the antigen is captured and detected by a sandwich assay as shown. Polyclonal antibodies are Protein G purified from rabbit immunized with NadA (TIGR-961) and attached to the bottom of the well. The bacterial sample is added to the well and allowed to bind. Biotinylated α-NadA antibody is added and then detected (using streptavidin-horseradish peroxidase (SA-HRP) with o-Phenylenediamine (OPD) substrate in the example in this Figure).

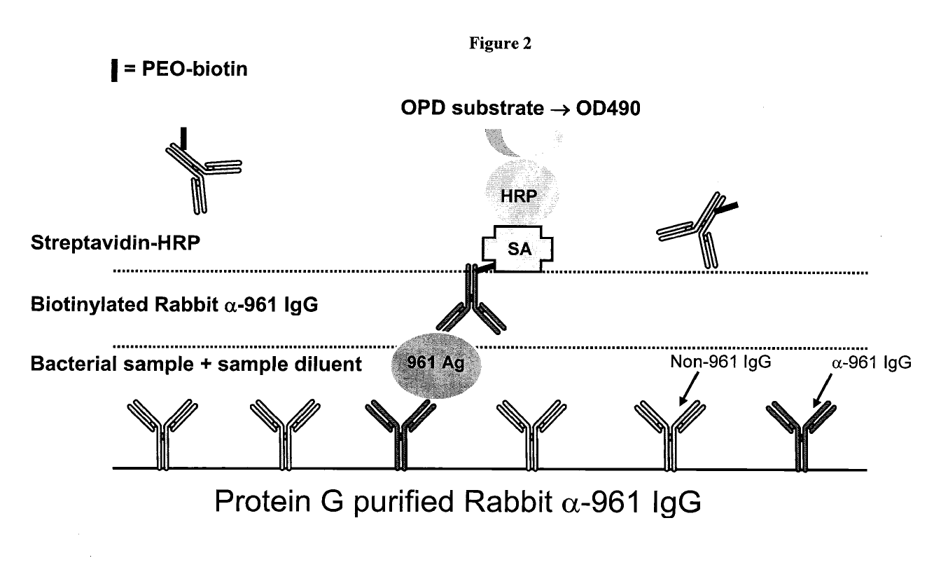

FIG. 3 shows the results obtained using anti-NadA (TIGR-961) antibodies ELISA (OD measured at 490 nm in 96-well plates) on bacteria samples prepared using different detergents (0.5% N-laurylsarcosine, 2% TRITON X-100™, 0.25% SB 3-14, and 0.5% EMPIGEN™ BB) in the preparation of samples of six different strains—three positive controls (5/99, GB364, 2996) and three negative controls (MC58, NZ98/254, E. coli). The results of the SBA are shown along the bottom of the figure by strain.

FIG. 4 shows the results of ELISAs on a NadA positive strain 5/99 using rabbit α-NadA antibodies with α-p24 antibodies and no sample as controls demonstrating the specificity of the ELISA assays.

FIG. 5 shows the results of Whole Cell ELISAs on two GNA1870 (TIGR-741) positive strains (MC58 and NZ98/254) and two GNA1870 negative strains (GB364 and 2996) with the correlated SBA results show below.

FIG. 6 shows the results of linear fit of the OD₄₉₂ versus concentration of recombinant GNA1870 (TIGR-741).

FIG. 7 shows the results of polynomial fit (five parameter logistic) of the OD₄₉₂ versus concentration of recombinant GNA1870 (TIGR-741).

FIG. 8 shows the results of linear fit of the OD₄₉₂ versus concentration of recombinant NadA (TIGR-961).

FIG. 9 shows the results of polynomial fit (five parameter logistic) of the OD₄₉₂ versus concentration of recombinant NadA (TIGR-961).

FIG. 10 compares the standard curves of OD₄₉₂ versus log dilution of recombinant NadA (TIGR-961) versus reference N. meningitidis strain 5/99. The box high-lights the divergence between the reference bacteria and the recombinant protein standard curves demonstrating that the reference bacteria are better as reference curve/calibrator.

### DETAILED DESCRIPTION OF THE PREFERRED EMBODIMENTS

The invention provides assays and methods, compositions and kits for performing such, for assessing efficacy of a vaccine against a pathogen whereby antibodies from a subject inoculated with a vaccine, a component of the vaccine or an epitope of the vaccine are tested for the ability to bind the pathogen or a component of the pathogen corresponding to the vaccine, a component of the vaccine or an epitope of the vaccine without performance of a functional assay.

### DEFINITIONS

A functional assay as used herein is any assay which measures a function of an antibody other than the ability of the antibody to bind to antigen. Examples of functional assays are serum bactericidal assays, opsonophagocytic assays, virus neutralization assays, and hemagglutination inhibition assays.

As used herein, polyclonal antibodies being directed to a vaccine, a vaccine component, a protein antigen, etc. refers to the polyclonal antibodies having been generated by inoculation of a subject with the item against which the polyclonal antibody is directed. One of skill in the art will recognize that polyclonal antibodies directed against an entire vaccine will not necessarily include antibodies that bind to every component of the vaccine as not all of the components are necessarily immunogenic. Further, such polyclonal antibodies may be further purified by affinity purification with a specific component or antigen to generate a subpopulation of antibodies that are specific to the component or antigen.

Vaccines

The methods and compositions disclosed herein may be applied to any vaccines as long as a correlation may be established between the binding of serum antibodies to the components of the vaccine as found in the pathogen of interest. The following embodiments are exemplary of the vaccines that may be assayed using the disclose methods and compositions. Where particular components are mentioned such as capsular polysaccharides or protein antigens, one of skill in the art will understand as discussed more fully in the detection antibodies below, that the assays disclosed herein may be performed using a combined sample of antibodies that detect all of the components of the vaccine such as would be produced by a subject inoculated with the whole vaccine by way of example or may be performed using antibodies for detection of each component individually or a subset of the components such as would be produced by a subject inoculated with an individual component.

For vaccines comprising polynucleotides that express antigens, one of skill in the art would recognize that the antibodies used to detect the pathogen or component or epitope of the pathogen would be antibodies that bind the encoded antigen rather than the polynucleotide.

In certain embodiments, the vaccines assayed include capsular saccharides from at least two of serogroups A, C, W135 and Y of Neisseria meningitides. In other embodiments, such vaccines further comprise an antigen from one or more of the following: (a) N. meningitidis; (b) Haemophilus influenzae type B; Staphylococcus aureus, groups A and B streptococcus, pathogenic E. coli, and/or (c) Streptococcus pneumoniae.

In certain embodiments the vaccines assayed include serogroups C, W135 & Y of N. meningitides. In certain embodiments the vaccines assayed include serogroups A, C, W135 & Y of N. meningitides. In certain embodiments the vaccines assayed serogroups B, C, W135 & Y of N. meningitides. In certain embodiments the vaccines assayed include serogroups A, B, C, W135 & Y of N. meningitides. In certain embodiments the vaccines assayed include H. influenzae type B and serogroups C, W135 & Y of N. meningitides. In certain embodiments the vaccines assayed include H. influenzae type B and serogroups A, C, W135 & Y of N. meningitides. In certain embodiments the vaccines assayed include H. influenzae type B and serogroups B, C, W135 & Y of N. meningitides. In certain embodiments the vaccines assayed include H. influenzae type B and serogroups A, B, C, W135 & Y of N. meningitides. In certain embodiments the vaccines assayed S. pneumoniae and serogroups C, W135 & Y of N. meningitides. In certain embodiments the vaccines assayed include S. pneumoniae and serogroups A, C, W135 & Y of N. meningitides. In certain embodiments the vaccines assayed include S. pneumoniae and serogroups B, C, W135 & Y of N. meningitides. In certain embodiments the vaccines assayed include S. pneumoniae and serogroups A, B, C, W135 & Y of N. meningitides. In certain embodiments the vaccines assayed include H. influenzae type B, S. pneumoniae and serogroups C, W135 & Y of N. meningitides. In certain embodiments the vaccines assayed include H. influenzae type B, S. pneumoniae and serogroups A, C, W135 & Y of N. meningitides. In certain embodiments the vaccines formulations containing at least one compound of Formula (I) include H. influenzae type B, S. pneumoniae and serogroups B, C, W135 & Y of N. meningitides. In certain embodiments the vaccines assayed include H. influenzae type B, S. pneumoniae and serogroups A, B, C, W135 & Y of N. meningitidis.

The methods and compositions disclosed herein can be use to determine efficacy of vaccines for various animals subjects including mammals such as human and non-human subjects, including, for example, pocket pets, fowl, and the like according to conventional methods well-known to those skilled in the art. Preferred vaccines will be vaccines with protein components which may be either recombinantly expressed or obtained from the pathogenic organism.

The methods and compositions disclosed herein can be used to assess manufacture of a vaccine to verify that each batch manufactured demonstrates requisite efficacy

Suitable vaccines that may be assayed using the methods and compositions disclosed herein include, but are not limited to, any material that raises a humoral immune response. Suitable vaccines assayed can include live viral and bacterial antigens and inactivated viral, tumor-derived, protozoal, organism-derived, fungal, and bacterial antigens, toxoids, toxins, proteins, glycoproteins, peptides, and the like, numerous examples of which are described below.

Antigens. Preferred antigens for assaying include those listed below.

### A. Bacterial Antigens

Bacterial antigens suitable for assaying with the disclosed methods and compositions include proteins, lipoproteins, proteoglycans, polysaccharides, lipopolysaccharides, and outer membrane vesicles which may be isolated, purified or derived from a bacteria. In addition, bacterial antigens may include bacterial lysates and inactivated bacteria formulations. Bacteria antigens may be produced by recombinant expression. Bacterial antigens preferably include epitopes which are exposed on the surface of the bacteria during at least one stage of its life cycle. Bacterial antigens are preferably conserved across multiple serotypes. Bacterial antigens include antigens derived from one or more of the bacteria set forth below as well as the specific antigens examples identified below.

Neisseria meningitides: Meningitides antigens may include proteins (such as those identified in References 1-7), saccharides (including a polysaccharide, oligosaccharide or lipopolysaccharide), or outer-membrane vesicles (References 8, 9, 10, 11) purified or derived from N. meningitides serogroup such as A, C, W135, Y, and/or B. Meningitides protein antigens may be selected from adhesions, autotransporters, toxins, Fe acquisition proteins, and membrane associated proteins (preferably integral outer membrane protein).

Streptococcus pneumoniae: Streptococcus pneumoniae antigens may include a saccharide (including a polysaccharide or an oligosaccharide) and/or protein from Streptococcus pneumoniae. Saccharide antigens maybe selected from serotypes 1, 2, 3, 4, 5, 6B, 7F, 8, 9N, 9V, 1OA, HA, 12F, 14, 15B, 17F, 18C, 19A, 19F, 20, 22F, 23F, and 33F. Protein antigens may be selected from a protein identified in WO98/18931, WO98/18930, U.S. Pat. No. 6,699,703, U.S. Pat. No. 6,800,744, WO97/43303, and WO97/37026. Streptococcus pneumoniae proteins may be selected from the Poly Histidine Triad family (PhtX), the Choline Binding Protein family (CbpX), CbpX truncates, LytX family, LytX truncates, CbpX truncate-LytX truncate chimeric proteins, pneumolysin (Ply), PspA, PsaA, Sp128, SpIO1, Sp130, Sp125 or Sp133.

Streptococcus pyogenes (Group A Streptococcus): Group A Streptococcus antigens may include a protein identified in WO02/34771 or WO05/032582 (including GAS 40), fusions of fragments of GAS M proteins (including those described in WO02/094851, and Dale, Vaccine (1999) 17:193-200, and Dale, Vaccine 14(10): 944-948), fibronectin binding protein (Sfbl), Streptococcal heme-associated protein (Shp), and Streptolysin S (SagA).

Moraxella catarrhalis: Moraxella antigens include antigens identified in WO02/18595 and WO99/58562, outer membrane protein antigens (HMW-OMP), C-antigen, and/or LPS.

Bordetella pertussis: Pertussis antigens include pertussis holotoxin (PT) and filamentous haemagglutinin (FHA) from B. pertussis, optionally also combination with pertactin and/or agglutinogens 2 and 3 antigen.

Staphylococcus aureus: Staphylococcus aureus antigens include S. aureus type 5 and 8 capsular polysaccharides optionally conjugated to nontoxic recombinant Pseudomonas aeruginosa exotoxin A, such as StaphVAX™, or antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin).

Staphylococcus epidermis: S. epidermidis antigens include slime-associated antigen (SAA).

Clostridium tetani (Tetanus): Tetanus antigens include tetanus toxoid (TT), preferably used as a carrier protein in conjunction/conjugated with the compositions of the present invention.

Cornynebacterium diphtheriae (Diphtheria): Diphtheria antigens include diphtheria toxin, preferably detoxified, such as CRM₁₉₇. Additionally antigens capable of modulating, inhibiting or associated with ADP ribosylation are contemplated for combination/co-administration/conjugation with the compositions of the present invention. The diphtheria toxoids may be used as carrier proteins.

Haemophilus influenzae B (Hib): Hib antigens include Hib protein antigens and Hib saccharide antigens.

Pseudomonas aeruginosa: Pseudomonas antigens include endotoxin A, Wzz protein, P. aeruginosa LPS, more particularly LPS isolated from PAO1 (05 serotype), and/or Outer Membrane Proteins, including Outer Membrane Proteins F (OprF)/

Legionella pneumophila: Bacterial antigens may be derived from Legionella pneumophila.

Streptococcus agalactiae (Group B Streptococcus): Group B Streptococcus antigens include a protein or saccharide antigen identified in WO02/34771, WO03/093306, WO04/041157, or WO05/002619 (including proteins GBS 80, GBS 104, GBS 276 and GBS 322, and including saccharide antigens derived from serotypes Ia, Ib, Ia/c, II, III, IV, V, VI, VII and VIII).

Neiserria gonorrhoeae: Gonorrhoeae antigens include Por (or porin) protein, such as PorB (see Zhu et al, Vaccine (2004) 22:660-669), a transferring binding protein, such as TbpA and TbpB (See Price et al, Infection and Immunity (2004) 71(1):277-283), a opacity protein (such as Opa), a reduction-modifiable protein (Rmp), and outer membrane vesicle (OMV) preparations (see Plante et al, J Infectious Disease (2000) 182:848-855), also see e.g. WO99/24578, WO99/36544, WO99/57280, WO02/079243).

Chlamydia trachomatis: Chlamydia trachomatis antigens include antigens derived from serotypes A, B, Ba and C (agents of trachoma, a cause of blindness), serotypes L1, L2 & L3 (associated with Lymphogranuloma venereum), and serotypes, D-K. Chlamydia trachomas antigens may also include an antigen identified in WO00/37494, WO03/049762, WO03/068811, or WO05/002619, including PepA (CT045), LcrE (CT089), ArtJ (CT381), DnaK (CT396), CT398, OmpH-like (CT242), L7/L12 (CT316), OmcA (CT444), AtosS (CT467), CT547, Eno (CT587), HrtA (CT823), and MurG (CT761).

Treponema pallidum (Syphilis): Syphilis antigens include TmpA antigen.

Haemophilus ducreyi (causing chancroid): Ducreyi antigens include outer membrane protein (DsrA).

Enterococcus faecalis or Enterococcus faecium: Antigens include a trisaccharide repeat or other Enterococcus derived antigens provided in U.S. Pat. No. 6,756,361.

Helicobacter pylori: H. pylori antigens include Cag, Vac, Nap, HopX, HopY and/or urease antigen.

Staphylococcus saprophyticus: Antigens include the 160 kDa hemagglutinin of S. saprophyticus antigen.

Yersinia enterocolitica: Antigens include LPS (Infect Immun. 2002 August; 70(8): 4414).

E. coli: E. coli antigens may be derived from meningitis/sepsis-associated E. coli (MNEC) (including antigens disclosed in WO06/089264), uropathogenic E. coli. (UPEC) (including antigens disclosed in WO06/091517), enterotoxigenic E. coli (ETEC), enteroaggregative E. coli (EAggEC), diffusely adhering E. coli (DAEC), enteropathogenic E. coli (EPEC), and/or enterohemorrhagic E. coli (EHEC).

Bacillus anthracis (anthrax): B. anthracis antigens are optionally detoxified and may be selected from A-components (lethal factor (LF) and edema factor (EF)), both of which can share a common B-component known as protective antigen (PA).

Yersinia pestis (plague): Plague antigens include F1 capsular antigen, LPS, and Yersinia pestis V antigen).

Mycobacterium tuberculosis: Tuberculosis antigens include lipoproteins, LPS, BCG antigens, a fusion protein of antigen 85B (Ag85B) and/or ESAT-6 optionally formulated in cationic lipid vesicles (Infect Immun. 2004 October; 72(10): 6148), Mycobacterium tuberculosis (Mtb) isocitrate dehydrogenase associated antigens, and/or MPT51 antigens (Infect Immun. 2004 July; 72(7): 3829).

Rickettsia: Antigens include outer membrane proteins, including the outer membrane protein A and/or B (OmpB) (Biochim Biophys Acta. 2004 Nov. 1; 1702(2): 145), LPS, and surface protein antigen (SPA) (J Autoimmun. 1989 June; 2 Suppl: 81).

Listeria monocytogenes: Bacterial antigens may be derived from Listeria monocytogenes.

Chlamydia pneumoniae: Antigens include those identified in WO02/02606.

Vibrio cholerae: Antigens include proteinase antigens, LPS, particularly lipopolysaccharides of Vibrio cholerae II, O1 Inaba O-specific polysaccharides, V. cholera O139, antigens of IEM108 vaccine {Infect Immun. 2003 October; 71(10):5498-504), and/or Zonula occludens toxin (Zot).

Salmonella typhi (typhoid fever): Antigens include protein antigens and capsular polysaccharides preferably conjugates (Vi, i.e., vax-TyVi).

Borrelia burgdorferi (Lyme disease): Antigens include lipoproteins (such as OspA, OspB, Osp C and Osp D), other surface proteins such as OspE-related proteins (Erps), decorin-binding proteins (such as DbpA), and antigenically variable VI proteins, such as antigens associated with P39 and P13 (an integral membrane protein) VlsE Antigenic Variation Protein.

Porphyromonas gingivalis: Antigens include P. gingivalis outer membrane protein (OMP).

Klebsiella: Antigens include an OMP, including OMP A, or a polysaccharide optionally conjugated to tetanus toxoid.

Further bacterial antigens of the invention may be capsular antigens, polysaccharide antigens or protein antigens of any of the above. Further bacterial antigens may also include an outer membrane vesicle (OMV) preparation. When using an OMV, preferred detection antibodies may be raised against a dominant epitope on the OMV such as PorA in the case of N. meningitidis. Additionally, antigens include live, attenuated, and/or purified versions of any of the aforementioned bacteria. The antigens of the present invention may be derived from gram-negative or gram-positive bacteria. The antigens of the present invention may be derived from aerobic or anaerobic bacteria.

Additionally, any of the above bacterial-derived saccharides (polysaccharides, LPS, LOS or oligosaccharides) can be conjugated to another agent or antigen, such as a carrier protein (for example CRM₁₉₇). Such conjugation may be direct conjugation effected by reductive amination of carbonyl moieties on the saccharide to amino groups on the protein, as provided in U.S. Pat. No. 5,360,897 and Can J Biochem Cell Biol. 1984 May; 62(5):270-5. Alternatively, the saccharides can be conjugated through a linker, such as, with succinamide or other linkages provided in Bioconjugate Techniques, 1996 and CRC, Chemistry of Protein Conjugation and Cross-Linking, 1993.

### B. Viral Antigens

Viral antigens that may be assayed with the methods and compositions disclosed herein include inactivated (or killed) virus, attenuated virus, split virus formulations, purified subunit formulations, viral proteins which may be isolated, purified or derived from a virus, and Virus Like Particles (VLPs). Viral antigens may be derived from viruses propagated on cell culture or other substrate. Alternatively, viral antigens may be expressed recombinantly. Viral antigens preferably include epitopes which are exposed on the surface of the virus during at least one stage of its life cycle. Viral antigens are preferably conserved across multiple serotypes or isolates. Viral antigens include antigens derived from one or more of the viruses set forth below as well as the specific antigens examples identified below.

Orthomyxovirus: Viral antigens may be derived from an Orthomyxovirus, such as Influenza A, B and C. Orthomyxovirus antigens may be selected from one or more of the viral proteins, including hemagglutinin (HA), neuraminidase (NA), nucleoprotein (NP), matrix protein (M1), membrane protein (M2), one or more of the transcriptase components (PB1, PB2 and PA). Preferred antigens include HA and NA.

Influenza antigens may be derived from interpandemic (annual) flu strains. Alternatively influenza antigens may be derived from strains with the potential to cause pandemic a pandemic outbreak (i.e., influenza strains with new haemagglutinin compared to the haemagglutinin in currently circulating strains, or influenza strains which are pathogenic in avian subjects and have the potential to be transmitted horizontally in the human population, or influenza strains which are pathogenic to humans).

Paramyxoviridae viruses: Viral antigens may be derived from Paramyxoviridae viruses, such as Pneumoviruses (RSV), Paramyxoviruses (PIV) and Morbilliviruses (Measles).

Pneumovirus: Viral antigens may be derived from a Pneumovirus, such as Respiratory syncytial virus (RSV), Bovine respiratory syncytial virus, Pneumonia virus of mice, and Turkey rhinotracheitis virus. Preferably, the Pneumovirus is RSV. Pneumovirus antigens may be selected from one or more of the following proteins, including surface proteins Fusion (F), Glycoprotein (G) and Small Hydrophobic protein (SH), matrix proteins M and M2, nucleocapsid proteins N, P and L and nonstructural proteins NS1 and NS2. Preferred Pneumovirus antigens include F, G and M. See e.g., J Gen Virol. 2004 November; 85(Pt 11):3229). Pneumovirus antigens may also be formulated in or derived from chimeric viruses. For example, chimeric RSV/PIV viruses may comprise components of both RSV and PIV.

Paramyxovirus: Viral antigens may be derived from a Paramyxovirus, such as Parainfluenza virus types 1-4 (PIV), Mumps, Sendai viruses, Simian virus 5, Bovine parainfluenza virus and Newcastle disease virus. Preferably, the Paramyxovirus is PIV or Mumps. Paramyxovirus antigens may be selected from one or more of the following proteins: Hemagglutinin-Neuraminidase (HN), Fusion proteins F1 and F2, Nucleoprotein (NP), Phosphoprotein (P), Large protein (L), and Matrix protein (M). Preferred Paramyxovirus proteins include HN, F1 and F2. Paramyxovirus antigens may also be formulated in or derived from chimeric viruses. For example, chimeric RSV/PIV viruses may comprise components of both RSV and PIV. Commercially available mumps vaccines include live attenuated mumps virus, in either a monovalent form or in combination with measles and rubella vaccines (MMR).

Morbillivirus: Viral antigens may be derived from a Morbillivirus, such as Measles. Morbillivirus antigens may be selected from one or more of the following proteins: hemagglutinin (H), Glycoprotein (G), Fusion factor (F), Large protein (L), Nucleoprotein (NP), Polymerase phosphoprotein (P), and Matrix (M). Commercially available measles vaccines include live attenuated measles virus, typically in combination with mumps and rubella (MMR).

Picornavirus: Viral antigens may be derived from Picornaviruses, such as Enteroviruses, Rhinoviruses, Heparnavirus, Cardioviruses and Aphthoviruses. Antigens derived from Enteroviruses, such as Poliovirus are preferred.

Enterovirus: Viral antigens may be derived from an Enterovirus, such as Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 and Enterovirus 68 to 71. Preferably, the Enterovirus is poliovirus. Enterovirus antigens are preferably selected from one or more of the following Capsid proteins VP1, VP2, VP3 and VP4. Commercially available polio vaccines include Inactivated Polio Vaccine (IPV) and Oral poliovirus vaccine (OPV).

Heparnavirus: Viral antigens may be derived from an Heparnavirus, such as Hepatitis A virus (HAV). Commercially available HAV vaccines include inactivated HAV vaccine.

Togavirus: Viral antigens may be derived from a Togavirus, such as a Rubivirus, an Alphavirus, or an Arterivirus. Antigens derived from Rubivirus, such as Rubella virus, are preferred. Togavirus antigens may be selected from E1, E2, E3, C, NSP-1, NSPO-2, NSP-3 or NSP-4. Togavirus antigens are preferably selected from E1, E2 or E3. Commercially available Rubella vaccines include a live cold-adapted virus, typically in combination with mumps and measles vaccines (MMR).

Flavivirus: Viral antigens may be derived from a Flavivirus, such as Tick-borne encephalitis (TBE), Dengue (types 1, 2, 3 or 4), Yellow Fever, Japanese encephalitis, West Nile encephalitis, St. Louis encephalitis, Russian spring-summer encephalitis, Powassan encephalitis. Flavivirus antigens may be selected from PrM, M, C, E, NS-I, NS-2a, NS2b, NS3, NS4a, NS4b, and NS5. Flavivirus antigens are preferably selected from PrM, M and E. Commercially available TBE vaccine include inactivated virus vaccines.

Pestivirus: Viral antigens may be derived from a Pestivirus, such as Bovine viral diarrhea (BVDV), Classical swine fever (CSFV) or Border disease (BDV).

Hepadnavirus: Viral antigens may be derived from a Hepadnavirus, such as Hepatitis B virus. Hepadnavirus antigens may be selected from surface antigens (L, M and S), core antigens (HBc, HBe). Commercially available HBV vaccines include subunit vaccines comprising the surface antigen S protein.

Hepatitis C virus: Viral antigens may be derived from a Hepatitis C virus (HCV). (see, e.g., Hsu et al. (1999) Clin Liver Dis 3:901-915). HCV antigens may be selected from one or more of E1, E2, E1/E2, NS345 polyprotein, NS 345-core polyprotein, core, and/or peptides from the nonstructural regions (Houghton et al, Hepatology (1991) 14:381). For example, Hepatitis C virus antigens that may be used can include one or more of the following: HCV E1 and or E2 proteins, E1/E2 heterodimer complexes, core proteins and non-structural proteins, or fragments of these antigens, wherein the non-structural proteins can optionally be modified to remove enzymatic activity but retain immunogenicity {see, e.g., WO03/002065; WO01/37869 and WO04/005473).

Rhabdovirus: Viral antigens may be derived from a Rhabdovirus, such as a Lyssavirus (Rabies virus) and Vesiculovirus (VSV). Rhabdovirus antigens may be selected from glycoprotein (G), nucleoprotein (N), large protein (L), nonstructural proteins (NS). Commercially available Rabies virus vaccine comprise killed virus grown on human diploid cells or fetal rhesus lung cells.

Caliciviridae: Viral antigens may be derived from Calciviridae, such as Norwalk virus, and Norwalk-like Viruses, such as Hawaii Virus and Snow Mountain Virus.

Coronavirus: Viral antigens may be derived from a Coronavirus, SARS, Human respiratory coronavirus, Avian infectious bronchitis (IBV), Mouse hepatitis virus (MHV), and Porcine transmissible gastroenteritis virus (TGEV). Coronavirus antigens may be selected from spike (S), envelope (E), matrix (M), nucleocapsid (N), and Hemagglutinin-esterase glycoprotein (HE). Preferably, the Coronavirus antigen is derived from a SARS virus. SARS viral antigens are described in WO04/92360.

Retrovirus: Viral antigens may be derived from a Retrovirus, such as an Oncovirus, a Lentivirus or a Spumavirus. Oncovirus antigens may be derived from HTLV-1, HTLV-2 or HTLV-5. Lentivirus antigens may be derived from HIV-I or HIV-2. Retrovirus antigens may be selected from gag, pol, env, tax, tat, rex, rev, nef, vif, vpu, and vpr. HIV antigens may be selected from gag (p24gag and p55gag), env (gp160 and gp41), pol, tat, nef, rev vpu, miniproteins, (preferably p55 gag and gp140v delete). HIV antigens may be derived from one or more of the following strains: HIVIIIb, HIVSF2, HIVLVA, HIVLAI, HIVMN, HIV-1CM235, and HIV-1US4.

Reovirus: Viral antigens may be derived from a Reovirus, such as an Orthoreovirus, a Rotavirus, an Orbivirus, or a Coltivirus. Reovirus antigens may be selected from structural proteins λ1, λ2, λ3, μ1, μ2, σ1, σ2, or σ3, or nonstructural proteins λNS, μNS, or σ1s. Preferred Reovirus antigens may be derived from a Rotavirus. Rotavirus antigens may be selected from VP1, VP2, VP3, VP4 (or the cleaved product VP5 and VP8), NSP 1, VP6, NSP3, NSP2, VP7, NSP4, or NSP5. Preferred Rotavirus antigens include VP4 (or the cleaved product VP5 and VP8), and VP7.

Parvovirus: Viral antigens may be derived from a Parvovirus, such as Parvovirus B19. Parvovirus antigens may be selected from VP-I, VP-2, VP-3, NS-I and NS-2. Preferably, the Parvovirus antigen is capsid protein VP-2.

Delta hepatitis virus (HDV): Viral antigens may be derived HDV, particularly δ-antigen from HDV (see, e.g., U.S. Pat. No. 5,378,814).

Hepatitis E virus (HEV): Viral antigens may be derived from HEV.

Hepatitis G virus (HGV): Viral antigens may be derived from HGV.

Human Herpesvirus: Viral antigens may be derived from a Human Herpesvirus, such as Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8). Human Herpesvirus antigens may be selected from immediate early proteins (α), early proteins (β), and late proteins (γ). HSV antigens may be derived from HSV-I or HSV-2 strains. HSV antigens may be selected from glycoproteins gB, gC, gD and gH, fusion protein (gB), or immune escape proteins (gC, gE, or gl). VZV antigens may be selected from core, nucleocapsid, tegument, or envelope proteins. A live attenuated VZV vaccine is commercially available. EBV antigens may be selected from early antigen (EA) proteins, viral capsid antigen (VCA), and glycoproteins of the membrane antigen (MA). CMV antigens may be selected from capsid proteins, envelope glycoproteins (such as gB and gH), and tegument proteins.

Papovaviruses: Antigens may be derived from Papovaviruses, such as Papillomaviruses and Polyomaviruses. Papillomaviruses include HPV serotypes 1, 2, 4, 5, 6, 8, 11, 13, 16, 18, 31, 33, 35, 39, 41, 42, 47, 51, 57, 58, 63 and 65. Preferably, HPV antigens are derived from serotypes 6, 11, 16 or 18. HPV antigens may be selected from capsid proteins (L1) and (L2), or E1-E7, or fusions thereof. HPV antigens are preferably formulated into virus-like particles (VLPs). Polyomyavirus viruses include BK virus and JK virus. Polyomavirus antigens may be selected from VP1, VP2 or VP3.

Further provided are antigens, compositions, methods, and microbes included in Vaccines, 4th Edition (Plotkin and Orenstein ed. 2004); Medical Microbiology 4th Edition (Murray et al. ed. 2002); Virology, 3rd Edition (W. K. Joklik ed. 1988); Fundamental Virology, 2nd Edition (B. N. Fields and D. M. Rnipe, eds. 1991), which are contemplated as assayable using the methods and compositions disclosed herein.

### C. Fungal Antigens

Fungal antigens that may be assayed with the methods and compositions disclosed herein may be derived from one or more of the fungi set forth below.

Fungal antigens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme.

Fungal pathogens may be derived from Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Processes for producing fungal antigens are well known in the art (see U.S. Pat. No. 6,333,164). In a preferred method a solubilized fraction extracted and separated from an insoluble fraction obtainable from fungal cells of which cell wall has been substantially removed or at least partially removed, characterized in that the process comprises the steps of: obtaining living fungal cells; obtaining fungal cells of which cell wall has been substantially removed or at least partially removed; bursting the fungal cells of which cell wall has been substantially removed or at least partially removed; obtaining an insoluble fraction; and extracting and separating a solubilized fraction from the insoluble fraction.

### D. STD Antigens

Additional compositions that may be assayed with the methods and compositions disclosed herein include one or more antigens derived from a sexually transmitted disease (STD). Such antigens may provide for prophylactis or therapy for STD's such as chlamydia, genital herpes, hepatitis (such as HCV), genital warts, gonorrhoea, syphilis and/or chancroid (See, WO00/15255). Antigens may be derived from one or more viral or bacterial STD's. Viral STD antigens for use in the invention may be derived from, for example, HIV, herpes simplex virus (HSV-1 and HSV-2), human papillomavirus (HPV), and hepatitis (HCV). Bacterial STD antigens for use in the invention may be derived from, for example, Neiserria gonorrhoeae, Chlamydia trachomatis, Treponema pallidum, Haemophilus ducreyi, E. coli, and Streptococcus agalactiae. Examples of specific antigens derived from these pathogens are described above.

### E. Respiratory Antigens

Additional compositions that may be assayed with the methods and compositions disclosed herein include one or more antigens derived from a pathogen which causes respiratory disease. For example, respiratory antigens may be derived from a respiratory virus such as Orthomyxoviruses (influenza), Pneumovirus (RSV), Paramyxovirus (PIV), Morbillivirus (measles), Togavirus (Rubella), VZV, and Coronavirus (SARS). Respiratory antigens may be derived from a bacteria which causes respiratory disease, such as Streptococcus pneumoniae, Pseudomonas aeruginosa, Bordetella pertussis, Mycobacterium tuberculosis, Mycoplasma pneumoniae, Chlamydia pneumoniae, Bacillus anthracis, and Moraxella catarrhalis. Examples of specific antigens derived from these pathogens are described above.

### F. Pediatric Vaccine Antigens

Additional compositions that may be assayed with the methods and compositions disclosed herein include one or more antigens suitable for use in pediatric subjects. Applying the methods and assays disclosed herein to pediatric vaccines, antibodies used will need to be correlated to efficacy in the pediatric subjects and may have been obtained from similar pediatric subjects or a model organism for such pediatric subjects. Pediatric subjects are typically less than about 3 years old, or less than about 2 years old, or less than about 1 years old. Pediatric antigens may be derived from a virus which may target pediatric populations and/or a virus from which pediatric populations are susceptible to infection. Pediatric viral antigens include antigens derived from one or more of Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), and Varicella-zoster virus (VZV), Epstein Barr virus (EBV). Pediatric bacterial antigens include antigens derived from one or more of Streptococcus pneumoniae, Neisseria meningitides, Streptococcus pyogenes (Group A Streptococcus), Moraxella catarrhalis, Bordetella pertussis, Staphylococcus aureus, Clostridium tetani (Tetanus), Cornynebacterium diphtheriae (Diphtheria), Haemophilus influenzae B (Hib), Pseudomonas aeruginosa, Streptococcus agalactiae (Group B Streptococcus), and E. coli. Examples of specific antigens derived from these pathogens are described above.

### G. Antigens Suitable for Use in Elderly or Immunocompromised Individuals

Additional compositions that may be assayed with the methods and compositions disclosed herein include one or more antigens suitable for use in elderly or immunocompromised individuals. Antigens which assayed for efficacy in Elderly or Immunocompromised individuals include antigens derived from one or more of the following pathogens: Neisseria meningitides, Streptococcus pneumoniae, Streptococcus pyogenes (Group A Streptococcus), Moraxella catarrhalis, Bordetella pertussis, Staphylococcus aureus, Staphylococcus epidermis, Clostridium tetani (Tetanus), Cornynebacterium diphtheriae (Diphtheria), Haemophilus influenzae B (Hib), Pseudomonas aeruginosa, Legionella pneumophila, Streptococcus agalactiae (Group B Streptococcus), Enterococcus faecalis, Helicobacter pylori, Clamydia pneumoniae, Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), Varicella-zoster virus (VZV), Epstein Barr virus (EBV), Cytomegalovirus (CMV). Examples of specific antigens derived from these pathogens are described above.

### H. Antigens Suitable for Use in Adolescent Vaccines

Additional compositions that may be assayed with the methods and compositions disclosed herein include one or more antigens suitable for use in adolescent subjects. Pediatric antigens which may be suitable for assay for efficacy in adolescents are described above. In addition, adolescents may be targeted to receive antigens derived from an STD pathogen in order to ensure protective or therapeutic immunity before the beginning of sexual activity. STD antigens which may be suitable for use in adolescents are described above.

### I. Tumor Antigens

### ANTIGEN REFERENCES

The following references include antigens that may be assayed with the methods and compositions disclosed herein:
1. WO99/24578
2. WO99/36544.
3. WO99/57280.
4. WO00/22430.
5. Tettelin et al. (2000) Science 287:1809-1815.
6. WO96/29412.
7. Pizza et al. (2000) Science 287:1816-1820.
8. WO01/52885.
9. Bjune et al. (1991) Lancet 338(8775).
10. Fuskasawa et al. (1999) Vaccine 17:2951-2958.
11. Rosenqist et al. (1998) Dev. Biol. Strand 92:323-333.
12. Constantino et al. (1992) Vaccine 10:691-698.
13. Constantino et al. (1999) Vaccine 17:1251-1263.
14. Watson (2000) Pediatr Infect Dis J 19:331-332.
15. Rubin (20000) Pediatr Clin North Am 47:269-285, v.
16. Jedrzejas (2001) Microbiol Mol Biol Rev 65:187-207.
17. WO02/02606.
18. Kalman et al. (1999) Nature Genetics 21:385-389.
19. Read et al. (2000) Nucleic Acids Res 28:1397-406.
20. Shirai et al. (2000) J. Infect. Dis 181(Suppl 3):S524-S527.
21. WO99/27 105.
22. WO00/27994.
23. WO00/37494.
24. WO99/28475.
25. Bell (2000) Pediatr Infect Dis J 19:1 187-1 188.
26. Iwarson (1995) APMIS 103:321-326.
27. Gerlich et al. (1990) Vaccine 8 Suppl: S63-68 & 79-80.
28. Hsu et al. (1999) Clin Liver Dis 3:901-915.
29. Gastofsson et al. (1996) N. Engl. J. Med. 334-:349-355.
30. Rappuoli et al. (1991) TIBTECH 9:232-238.
31. Vaccines (1988) eds. Plotkin & Mortimer. ISBN 0-7216-1946-0.
32. Del Guidice et al. (1998) Molecular Aspects of Medicine 19:1-70.
33. WO93/018150.
34. WO99/533 10.
35. WO98/04702.
36. Ross et al. (2001) Vaccine 19:135-142.
37. Sutter et al. (2000) Pediatr Clin North Am 47:287-308.
38. Zimmerman & Spann (1999) Am Fan Physician 59:1 13-1 18, 125-126.
39. Dreensen (1997) Vaccine 15 Suppl S2-6.
40. MMWR Morb Mortal Wkly rep 1998 Jan. 16: 47(1): 12, 9.
41. McMichael (2000) Vaccine 19 Suppl 1:S101-107.
42. Schuchat (1999) Lancer 353(9146):5 1-6.
43. GB patent applications 0026333.5, 0028727.6 & 0105640.7.
44. Dale (1999) Infect Disclin North Am 13:227-43, viii.
45. Ferretti et al. (2001) PNAS USA 98: 4658-4663.
46. Kuroda et al. (2001) Lancet 357(9264): 1225-1240; see also pages 1218-1219.
47. Ramsay et al. (2001) Lancet 357(9251): 195-196.
48. Lindberg (1999) Vaccine 17 Suppl 2:S28-36.
49. Buttery & Moxon (2000) J R Coil Physicians Long 34: 163-168.
50. Ahmad & Chapnick (1999) Infect Dis Clin North Am 13:1 13-133, vii.
51. Goldblatt (1998) J. Med. Microbiol. 47:663-567.
52. EP Patent 0477508.
53. U.S. Pat. No. 5,306,492.
54. WO98/42721.
55. Conjugate Vaccines (eds. Cruse et al.) ISBN 3805549326, particularly vol. 10.48-1 14.
56. Hermanson (1996) Bioconjugate Techniques ISBN: 012323368 & 012342335X.
57. EP Patent Appl. 0372501.
58. EP Patent Appl. 0378881.
59. EP Patent Appl. 0427347.
60. WO93/17712.
61. WO98/58668.
62. EP Patent Appl. 0471177.
63. WO00/56360.
64. WO00/67161.

N. Meningitidis Serogroup B

A preferred pathogen is N. meningitidis serogroup B. Two examples of preferred vaccine for N. meningitidis serogroup B are (i) a five component vaccine comprising three primary components: NadA, GNA1870 and GNA2132; and two accessory components GNA1030 and GNA2091. In certain embodiments, the accessory components may be fused to the primary components, preferably GNA1030 is fused to the C-terminus of GNA2132 and GNA1870 is fused to the C-terminus of GNA2091. Additional disclosure regarding the five component vaccine may be found in WO04/032958. In certain embodiments, the five component vaccine may be combined with a membrane preparation derived from a N. meningitidis serogroup B strain, preferably an OMV membrane preparation.

NadA antigens. ‘NadA’ (Neisserial adhesin A) from serogroup B of N. meningitidis is disclosed as protein ‘961’ in reference (R3) (SEQ IDs 2943 & 2944) and as ‘NMB1994’ in reference (R2) (see also GenBank accession numbers: 11352904 & 7227256). A detailed description of the protein can be found in reference (R9). There is no corresponding protein in serogroup A ((R1), (R9)).

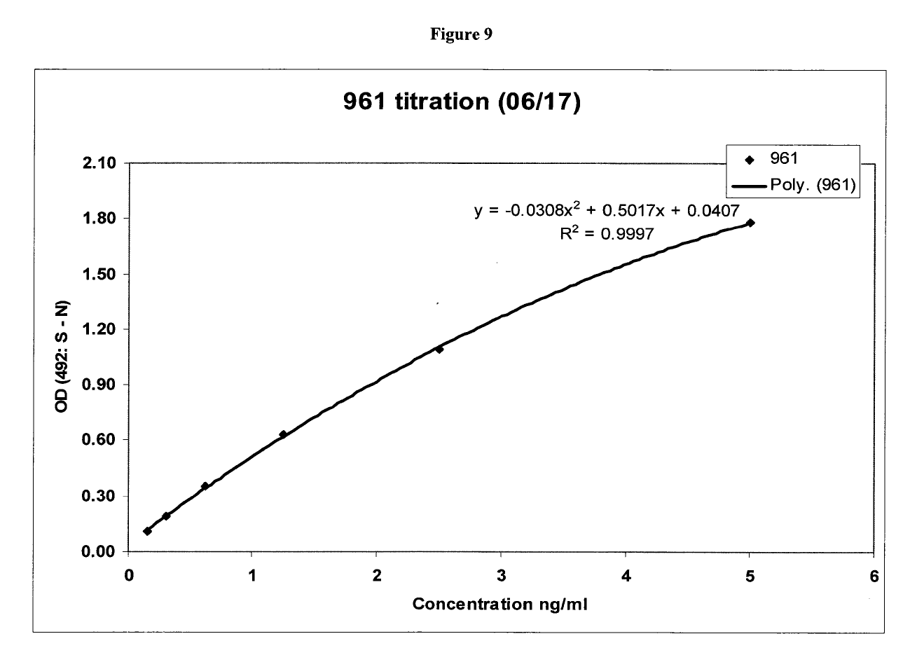

NadA may take various forms in vaccines. Preferred forms of NadA are truncation or deletion variants, such as those disclosed in references (R6), (R7), and (R8). In particular, NadA without its C terminal membrane anchor is preferred (e.g., deletion of residues 351 405 for strain 2996), which is sometimes distinguished herein by the use of a ‘C’ superscript, e.g., NadA^{(C)}. Expression of NadA without its membrane anchor domain in E. coli results in secretion of the protein into the culture supernatant with concomitant removal of its 23mer leader peptide (e.g., to leave a 327mer for strain 2996). Polypeptides without their leader peptides are sometimes distinguished herein by the use of a ‘NL’ superscript, e.g., NadA^{(NL)} or NadA^{(C)(NL)}. NadA occurs in three main allelic variants as shown in FIG. 9 of reference (R10).

Vaccines may also comprise fragments which comprise an epitope from NadA in which case, detection of the epitope in a pathogen of interest may be performed using a monoclonal antibody to the epitope.

Secreted NadA can conveniently be prepared in highly pure form from culture supernatant by a process comprising the steps of: concentration and diafiltration against a buffer by ultrafiltration; anionic column chromatography; hydrophobic column chromatography; hydroxylapatite ceramic column chromatography; diafiltration against a buffer; and filter sterilisation. Further details of the process are given in the examples.

NadA is preferably used in an oligomeric form (e.g., in trimeric form).

GNA1870 Antigens. ‘GNA1870’ protein from serogroup B is disclosed as protein ‘741’ in reference (R3) (SEQ IDs 2535 & 2536) and as ‘NMB1870’ in reference (R2) (see also GenBank accession number GI:7227128). The corresponding protein in serogroup A (R1) has GenBank accession number 7379322. GNA1870 is naturally a lipoprotein.

When as an antigen in a vaccine, GNA1870 protein may take various forms. Preferred forms of GNA1870 are truncation or deletion variants, such as those disclosed in references (R6), (R7), and (R8). In particular, the N terminus of GNA1870 may be deleted up to and including its poly-glycine sequence (i.e., deletion of residues 1 to 72 for strain MC58), which is sometimes distinguished herein by the use of a ‘ΔG’ prefix. This deletion can enhance expression. The deletion also removes GNA1870's lipidation site.

Allelic forms of GNA1870 may also be used as antigens and examples of alleles can be found in SEQ IDs 1 to 22 of reference (R8), and in SEQ IDs 1 to 23 of reference (R11). SEQ IDs 1-299 of reference (R12) give further GNA1870 sequences.

Vaccines may also comprise fragments which comprise an epitope from GNA1870 in which case, detection of the epitope in a pathogen of interest may be performed using a monoclonal antibody to the epitope.

Protein GNA1870 is an extremely effective antigen for eliciting anti meningococcal antibody responses, and it is expressed across all meningococcal serogroups. Phylogenetic analysis shows that the protein splits into two groups, and that one of these splits again to give three variants in total (R13), and while serum raised against a given variant is bactericidal within the same variant group, it is not active against strains which express one of the other two variants, i.e., there is intra-variant cross protection, but not inter variant cross protection. Through the use of monoclonal or polyclonal antibodies specific to one variant or another, one of skill in the art could differentiate between these groups. For maximum cross-strain efficacy, therefore, it is preferred that a vaccine should include more than one variant of protein GNA1870 and therefore the corresponding detection antibodies should take into account the nature of the vaccine. For example, a vaccine composition with one of each group will likely need at least a monoclonal antibody from each variant for detection.

GNA2091 Antigens. ‘GNA2091’ protein from serogroup B is disclosed as protein ‘936’ in reference (R3) (SEQ IDs 2883 & 2884) and as ‘NMB2091’ in reference (R2) (see also GenBank accession number GI:7227353). The corresponding gene in serogroup A (R1) has GenBank accession number 7379093.

When used as an antigen in a vaccine, GNA2091 protein may take various forms. Preferred forms of GNA2091 are truncation or deletion variants, such as those disclosed in references (R6), (R7), and (R8). In particular, the N terminus leader peptide of GNA2091 may be deleted (i.e., deletion of residues 1 to 23 for strain MC58) to give GNA2091^{(NL)}.

GNA2091 antigens may also include variants (e.g., allelic variants, homologs, orthologs, paralogs, mutants etc).

Vaccines may also comprise fragments which comprise an epitope from GNA2091 in which case, detection of the epitope in a pathogen of interest may be performed using a monoclonal antibody to the epitope.

GNA1030 Antigens. ‘GNA1030’ protein from serogroup B is disclosed in as ‘953’ in reference (R3) (SEQ IDs 2917 & 2918) and as ‘NMB1030’ in reference (R2) (see also GenBank accession number GI:7226269). The corresponding protein in serogroup A (R1) has GenBank accession number 7380108.

When used according to the present invention, GNA1030 protein may take various forms. Preferred forms of GNA1030 are truncation or deletion variants, such as those disclosed in references (R6), (R7), and (R8). In particular, the N terminus leader peptide of 953 may be deleted (i.e., deletion of residues 1 to 19 for strain MC58) to give 953^{(NL)}.

GNA1030 antigens may also include variants (e.g., allelic variants, homologs, orthologs, paralogs, mutants, etc.). Allelic forms of GNA1030 can be seen in FIG. 19 of reference (R4).

Vaccines may also comprise fragments which comprise an epitope from GNA11030 in which case, detection of the epitope in a pathogen of interest may be performed using a monoclonal antibody to the epitope.

GNA2132 Antigens. ‘287’ protein from serogroup B is disclosed as ‘287’ in reference (R3) (SEQ IDs 3103 & 3104), as ‘NMB2132’ in reference (R2), and in reference (R5) (see also GenBank accession number GI:7227388). The corresponding protein in serogroup A (R1) has GenBank accession number 7379057.

When used according to the present invention, GNA2132 protein may take various forms. Preferred forms of GNA2132 are truncation or deletion variants, such as those disclosed in references (R6), (R7), and (R8). In particular, the N terminus of GNA2132 may be deleted up to and including its poly glycine sequence (i.e., deletion of residues 1 to 24 for strain MC58), which is sometimes distinguished herein by the use of a ‘ΔG’ prefix. This deletion can enhance expression.

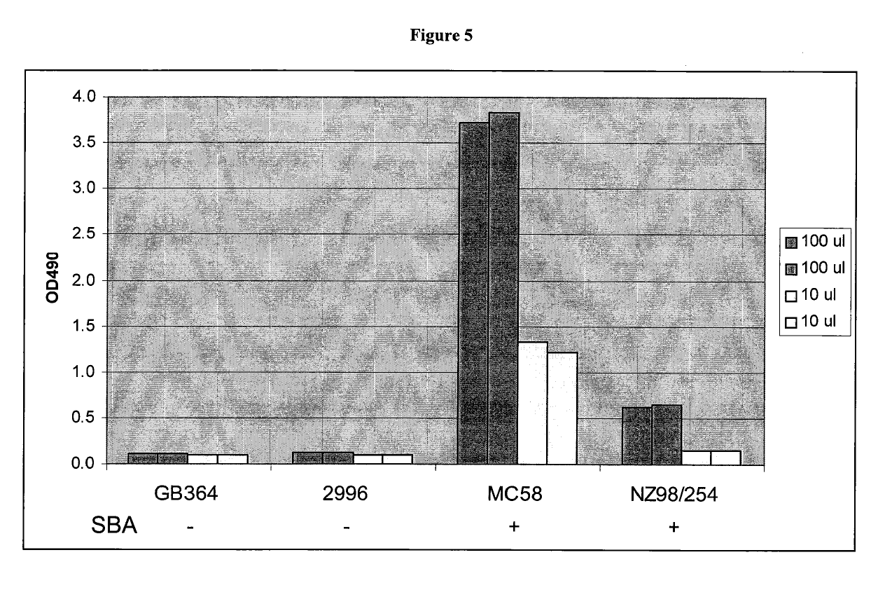

GNA2132 antigens may also include variants (e.g., allelic variants, homologs, orthologs, paralogs, mutants, etc.). Allelic forms of GNA2132 can be seen in FIGS. 5 and 15 of reference (R4), and in example 13 and FIG. 21 of reference (R3) (SEQ IDs 3179 to 3184).

Strains. Preferred antigens for N. meningitidis serogroup B vaccines are from strains 2996, MC58, 95N477, and 394/98. Strain 394/98 is sometimes referred to herein as ‘NZ’, as it is a New Zealand strain.

GNA2132 is preferably from strain 2996 or, more preferably, from strain 394/98.

GNA1870 is preferably from serogroup B strains MC58, 2996, 394/98, or 95N477, or from serogroup C strain 90/18311. Strain MC58 is more preferred.

Antigens GNA2091, GNA1030 and NadA are preferably from strain 2996.

Hypervirulent lineages and bactericidal antibody responses. In general, vaccines against N. meningitidis serogroup B will be able to induce serum bactericidal antibody responses after being administered to a subject which may be verified by testing in the assays disclosed herein without need of a functional assay such as the serum bactericidal assay.

Rather than offering narrow protection, vaccines against N. meningitidis serogroup B induce bactericidal antibody responses against more than one hypervirulent lineage of serogroup B; however, even within a particular hypervirulent lineage, a vaccine may not be effective against all strains in the lineage. Therefore one of skill in the art would want an assay such as disclosed herein to determine if a vaccine is effective against particular strain of interest before using the vaccine.

References for the N. meningitidis serogroup B vaccines:
(R1) Parkhill et al. (2000) Nature 404:502-506.
(R2) Tettelin et al. (2000) Science 287:1809-1815.
(R3) WO99/57280.
(R4) WO00/66741.
(R5) Pizza et al. (2000) Science 287:1816-1820.
(R6) WO01/64920.
(R7) WO01/64922.
(R8) WO03/020756.
(R9) Comanducci et al. (2002) J. Exp. Med. 195:1445-1454.
(R10) WO03/010194.
(R11) UK patent application 0227346.4.
(R12) WO03/063766.
(R13) Masignani et al. (2003) J Exp Med 197:789-799.

Detection Antibodies

The antibodies used in the methods and compositions disclosed herein may be obtained from any source so long as the binding of the antibody to a pathogen or component or epitope within the antigen can be correlated to a vaccine's efficacy against a pathogen as measured by a functional assay. Thus one of skill in the art would understand that the vaccine or any immunogenic component or epitope within the vaccine may be used to generate antibodies that may be used in the invention disclosed herein. In certain embodiments, the antibody preparation may be in the form of an antibody containing serum sample, polyclonal antibodies, antigen-purified polyclonal antibodies or monoclonal antibodies. The antibody preparation may bind to all immunogenic components of a vaccine, to one component of a multicomponent vaccine or to a specific epitope of a vaccine component.

Preparation of Pathogen Samples

The pathogen of interest may be assayed using the methods or compositions herein either as whole pathogen (i.e., whole cell in the case of bacteria, fungus or tumor or whole virus) or as a partially or wholly extracted or purified component wherein the component assayed is also a component of the vaccine for which efficacy is being assessed or assayed.

When extracting or purifying a component of a pathogen, one of skill in the art may use any techniques available for extraction or purification. By way of example, where the component is a protein antigen, one of skill in the art may use any protein extraction or purification technique. Where the protein antigen is a membrane bound or associated protein, a preferred embodiment would include use of a detergent to extract or solubilize the protein, preferably a zwittergent, more preferably EMPIGEN™ BB.

Assays for Detection of Binding

In order to assess or assay efficacy of a vaccine against a pathogen of interest, the ability of an antibody produced in a subject inoculated with the vaccine to be tested or a component or epitope of the vaccine to bind the pathogen or a component of the pathogen corresponding to the applicable component of the vaccine is detected using any technique available to one of skill in the art for detection of antibody binding which is not a functional assay. By way of example, detection methods include western-blot, ELISA, lateral flow assay, latex-agglutination, immunochromatographic strips, fluorescence (including multichannel flow cytometric fluorescence detection methods), rate nephelometry, or immuno-precipitation.

In certain embodiments, the antibodies may be fixed to a solid support such as a multi-well plate such as a 96 or 384-well plate, bead, sphere, membrane, colloidal metal (e.g. gold), porous member, surfaces of capillary (e.g. in flow through test), test strip or latex particle. In other embodiments the pathogen or the component or epitope of the pathogen may be affixed to such a solid support either directly or by indirect linkage such as a capture antibody as used in sandwich ELISA. Examples of direct linkage of an antibody to a solid support or an enzyme or other labeling moiety or a pathogen, or a component or epitope from such pathogen, to a support include covalent binding, non-covalent binding, or adsorption to a surface of the support or within the support in the case of a gel support such as agarose or acrylamide. Examples of direct linkage of an antibody to a solid support or an enzyme or other labeling moiety or a pathogen, or a component or epitope from such pathogen, to a support mediated by binding partners such as avidin-biotin, streptavidin-biotin, digioxingenin-anti-digoxigenin, antibody-epitope, etc.

When using ELISA based detection, any suitable assayable enzyme may be used including by way of example, horse-radish peroxidase, alkaline phosphatase, β-galactosidase, luciferase, and acetylcholinesterase. One of skill in the art may select any suitable substrate for the enzyme chosen such as a chromogenic, radiolabeled or a fluorescent substrate.

When assessing or assaying efficacy, the efficacy may be determined by any suitable method for analyses of the results of the particular antibody binding assay that may be correlated to the efficacy of the vaccine. In simple embodiments, the assay may produce a binary result such as a latex agglutination assay which is tuned such that no aggregation occurs when a vaccine is not efficacious against a pathogen while aggregation occurs when the vaccine is efficacious. In other embodiments, the analysis will produce a numerical value whereby a value above or below a threshold indicates efficacy. Preferred analysis methods with numerical output include the % Bₘₐₓ method as set forth in Example 6, below, and the signal-to-noise ratio (S/N) in which the signal from the pathogen sample is divided by the signal from the blank. For methods with numerical output a preferred embodiment would include a standard curve obtained with different concentrations of a reference preparation of antigen and testing of several different dilutions of the pathogen sample.

Kits

The methods and compositions disclosed herein may be embodied in a kit for the practice of the assays. In one aspect, the kits for use in methods and compositions as disclosed herein will include (a) an antibody sample that binds to the vaccine of interest or a component or epitope thereof, (b) a detection moiety comprising an enzyme which is linked to the antibody or may be linked to the antibody during the assay, and (c) a reagent for extraction of a component of interest from the pathogen to be tested. A preferred example would be a kit comprising three antibody samples which bind to GNA1870, GNA2132, and NadA, respectively.

General

The term “comprising” encompasses “including” as well as “consisting” e.g., a composition “comprising” X may consist exclusively of X or may include something additional e.g., X+Y.

The word “substantially” does not exclude “completely” e.g., a composition which is “substantially free” from Y may be completely free from Y. Where necessary, the word “substantially” may be omitted from the definition of the invention. The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Where a cell substrate is used for reassortment or reverse genetics procedures, it is preferably one that has been approved for use in human vaccine production e.g., as in Ph Eur general chapter 5.2.3.

Identity between polypeptide sequences is preferably determined by the Smith-Waterman homology search algorithm as implemented in the MPSRCH program (Oxford Molecular), using an affine gap search with parameters gap open penalty=12 and gap extension penalty=1.

### Example 1

### ELISA Assays for Efficacy of 961 and 741 Components Against a Pathogen Strain

This example provides a representative assay for determination of efficacy of two components of the multicomponent N. meningitidis serogroup B vaccine.

Antibody Production

Mice immunizations. To prepare antisera, 20 μg of individual antigens, GNA2132 (TIGR-287), GNA1030 (TIGR-953), GNA2091 (TIGR-936), GNA1870 (TIGR 741), NadA (GNA1994 or TIGR-961), GNA2132-1030, GNA2091-1870 or a combination of 20 μg of each of GNA2132-1030, GNA2091-1870 and NadA were used to immunize six-week-old CD1 female mice (Charles River). Eight to ten mice per group were used. The antigens were administered intraperitoneally (i.p.), together with aluminum hydroxide (3 mg/ml) on day 0, 21 and 35. Blood samples for analysis were taken on day 34 and on day 49.

Rabbit Immunizations. 50 μg of individual antigens, GNA2132, GNA1030, GNA2091, GNA1870, NadA, GNA2132-1030, GNA2091-1870 or a combination of 50 μg of each of GNA2132-1030, GNA2091-1870 and NadA were used to immunize New Zealand White rabbits. The antigens were administered subcutaneously (s.c.), together with aluminum hydroxide (3 mg/ml) on day 0, 21 and 35. Blood samples for analysis were taken on day 34 and with a final bleed on day 49.

Preparation of mAb 502. Four- to six-weeks-old female CD1 mice were immunized with 20 μg of variant 1 GNA1870 (TIGR-741) recombinant protein. The recombinant protein was administered intraperitoneally (i.p.), together with complete Freund's adjuvant (CFA) for the first dose and incomplete Freund's adjuvant (IFA) for the second (day 21) and without adjuvant for booster dose (day 35). Three days later, the mice were sacrificed and their spleen cells were fused with myeloma cells P3×63-Ag8.653 at a ratio of five spleen cells to one myeloma cell. After a two-week incubation in hypoxanthine/aminopterin/thymidine (HAT) selective medium, hybridoma supernatants were screened for Ab binding activity by ELISA performed on microtiter plates as following.

Variant 1 GNA1870 (1 μg/ml in PBS) was used to coat 96-well plates (Greiner), 100 μl per well. Coating wells with whole cell bacteria for the WCE was performed with 100 μl bacterial cell culture in PBS containing 0.025% formaldehyde (OD₆₂₀ 0.25-0.3) by overnight incubation at 4° C. Wells were washed three times with 300 μl of wash buffer (PBS containing 0.1% TWEEN 20™ (Polyoxyethylene (20) sorbitan monolaurate)) and then were saturated with 200 μl of saturation buffer (2.7% polyvinylpyrrolidone 10 in water). 100 μl of the hybridoma supernatant undiluted or a polyclonal mouse serum anti GNA 1870 (positive control) were added to each well and incubated for two hours at 37° C. Plates were washed three times with wash buffer. 100 μl of HRP conjugate rabbit anti-mouse (Sigma) diluted 1/2,000 with dilution buffer (1% BSA, 0.1% TWEEN 20™, in PBS), were added to each well and incubated for 1 hour and 30 minutes at 37° C. 100 μl of substrate buffer for HRP (25 ml of citrate buffer pH5, 10 mg of O-phenyldiamine and 10 μl of H₂O₂ 30%) were added to each well and incubated for 20 minutes, the reaction was stopped with 100 μl of sulphuric acid 12.5% v/v. ELISA titers were expressed as the reciprocal of the last dilution of sera or hybridoma supernatants, which gave an OD₄₉₀ value of 0.4. The ELISA titers were considered positive when the dilution of sera with OD₄₉₀ of 0.4 was higher than 1/400. Hybridomas secreting GNA1870-specific Ab were cloned twice by limiting dilution and then expanded and frozen for subsequent use in tissue culture, or for ascites production in BALB/c mice. The subclasses of the mAb were determined using a mouse mAb isotyping kit (Amersham Pharmacia Biotech). Among the selected mAbs, one IgG2a anti-GNA1870 mAb, designated mAb 502, was used in all the binding and functional assays in the following examples. This mAb was purified from mouse ascites by HiTrap™ affinity columns (Amersham Pharmacia Biotech) and, after exhaustive dialysis in PBS buffer, the concentration of the purified mAb was determined using a modified Lowry method with BSA as a standard (Bio-Rad DC Protein Assay; Bio-Rad, München, Germany). Specificity of mAb502 binding was determined by Western blot and FACS analysis.

Sample Preparation

The pathogen sample was prepared as follows:
1. 25 μl of specimen diluent (5% EMPIGEN BB™ (n-Dodecyl-N,N-dimethylglycine), 0.25% ProClin™ 300, 0.01% methylene blue in 0.1 M phosphate buffered 1.5 M NaCl, pH 7.4) at room temperature was pipetted into a 1.5 mL microcentrifuge tube.
2. 250 μl of N. meningitidis serogroup B in culture broth was added to the tube. (OD₆₀₀ ˜0.3-0.5).
3. The tube was vortexed to mix and allowed to incubate at room temperature for 30 min.

The ELISA Protocol was performed as follows:
1) Bring all reagents to RT before use.
2) Set up ELISA plate allowing 2 wells for negative controls and 2 wells for positive controls. Well 1A should be left empty for use as a blank (no solutions except for substrate in Step 16).
3) Add 100 μl of negative control (Culture broth without bacteria plus sample diluent) to 2 wells.
4) Add 100 μl of positive control (Bacterial suspension against which the vaccine was known to be efficacious) with to 2 wells.
5) Add 110 μl of MenB strain samples prepared as above to the appropriate wells.
6) Add 10 μl of specimen diluent (as above) to the positive and negative controls.
7) Apply plate cover sealer. Shake for 30 sec. on an orbital shaker to mix.
8) Incubate for 1 hour @ 37° C.
9) Remove plate cover sealer and wash plate 4 times with 350 μl of ELISA wash buffer (PBS with 0.05% TWEEN 20™); basically—fill the wells with wash buffer). Tap plate on paper towel to remove excess wash buffer.
10) Add 100 μl of Biotin-Ab (20% normal rabbit serum, 0.25% ProClin™ 300, 0.1% BSA in 50 mM Tris buffer containing 0.15 M NaCl and 0.05% TWEEN 20™, 1 microgram/mL of Ab) solution to each well.
11) Apply plate cover sealer. Incubate for 1 hour @ 37° C.
12) Remove plate cover sealer and wash plate 4 times with 350 μl of ELISA wash buffer. Tap plate on paper towel to remove excess wash buffer.
13) Add 100 μl of Streptavidin-HRP solution (20% normal rabbit serum, 0.25% ProClin™300, 0.1% BSA, trace potassium ferricyanide, 1 0.25 microgram/mL streptavidin-HRP in 50 mM Tris buffer with 0.15N NaCl and 0.05% TWEEN 20™) to each well.
14) Apply plate cover. Incubate for 1 hour @ 37° C. (May also be incubated for 30 min. @ 37° C.)
15) Remove plate cover sealer and wash plate 4 times with 350 μl of ELISA wash buffer. Tap plate on paper towel to remove excess wash buffer.
16) Add 100 μl of OPD substrate solution (add 1 OPD tablet (Sigma P8287) dissolved in 6 ml of substrate buffer) to each well, including blank well A1.
17) Incubate at RT in the dark for 20 min.
18) Add 50 μl of 4N H2SO4 to stop the reaction.
19) Read the OD at 492 nm (or 490 is OK).

In addition to reading the plate at a single wavelength, a preferred method is to run a blank in well A1 (See step 2 above), read the plate at dual wavelengths (read 492 nm with reference >600 nm) and subtract the blank value from your other OD values. Steps 10-16 may be simplified by pre-incubation of the biotin conjugated antibody and the Streptavidin-HRP or by use of HRP or other enzyme directly coupled to the antibody. In alternate embodiments,

### Example 2

### Comparison of Different Detergents for Sample Preparation

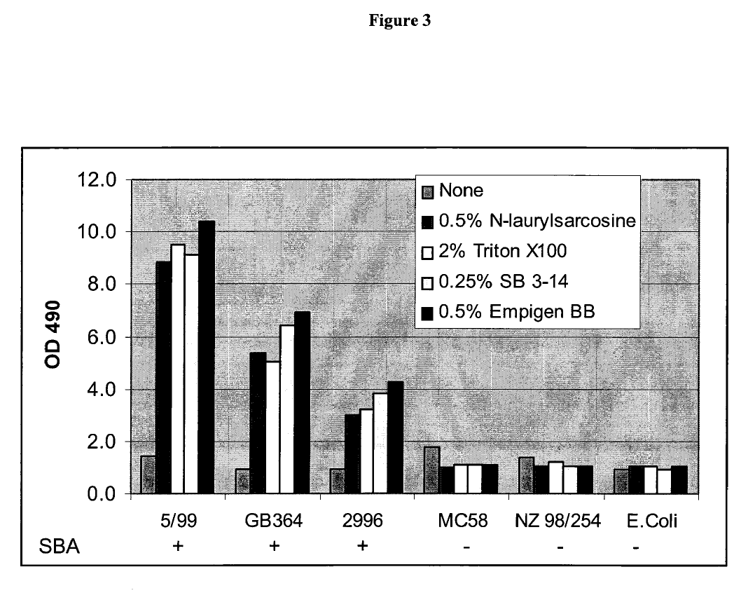

The following example demonstrates use of the protocol set out in Example 1 to compare different detergents in sample preparation for assaying efficacy against NadA. 96 well plates were coated with rabbit anti-NadA polyclonal antibodies. The assays were performed as set forth in Example 1 except that five different sample diluents were compared. Diluent without detergent was prepared as a negative control. Two diluents were prepared with zwitterionic detergents—5% EMPIGEN™ BB and 2.5% SB 3-14 (n-Tetradecyl-N,N-dimethyl-3-ammonio-1-propanesulfonate). One diluent was prepared with an anionic detergents—5% N-laurylsarcosine. One diluent was prepared with a nonionic detergent—20% TRITON X-100™. Each of the detergents solubilized the NadA membrane protein sufficiently to detect in the ELISA to correlate to the SBA results as shown in FIG. 3.

### Example 3

### Comparison of Ag ELISA and Whole Cell ELISA

The Whole Cell ELISA for the NadA antigen was performed as in Example 1, modified to account for the bacterial cells being coated on the 96-well plate directly rather than added after solubilization with the sample diluent (i.e., omitting step 5). The results of the Whole Cell ELISA are provided in Table 1 below.

TABLE 1
Comparison of SBA, WCE and Ag ELISA for NadA

| | | WCE | Ag ELISA |
|---|---|---|---|
| MenB Strain | SBA Results | Results S/N | Results S/N |
| 5/99 (NadA+) | + | 9.3 | >20 |
| GB364 (NadA+) | + | 4.8 | 6.9 |
| 2996 (NadA+) | + | 3.3 | 4.3 |
| MC58 (NadA+) | − | 1.0 | 1.1 |
| NZ98/254 (NadA−) | − | 0.8 | 1.1 |

As shown in Table 1, the WCE and the Ag ELISA both correlated to the SBA results.

### Example 4

### Demonstration of the Specificity ELISA

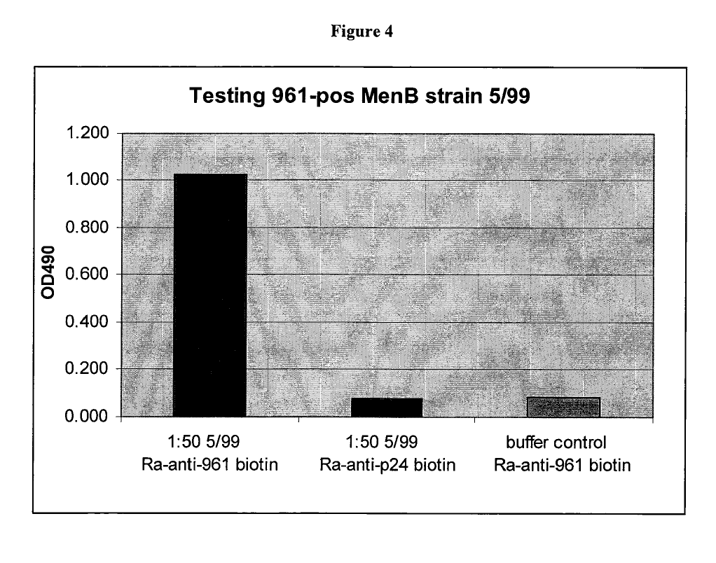

To demonstrate the specificity of the ELISA assays, three ELISAs were run as set forth in Example 1 where the first assay and second assays used a NadA positive strain 5/99 while the third assay had no bacteria. The first and third assays used rabbit α-NadA antibodies for capture and detection while the second assay used α-p24 (a non-N. meningitidis antigen) antibodies. Only the first assay showed any signal as shown in FIG. 4 demonstrating that the assay shows the specificity that one of skill in the art would expect.

### Example 5

### Extension to GNA1870

To demonstrate the applicability to other antigens, Whole Cell ELISAs were performed using 96-well plates with formalin fixed in accordance with the modified protocol of Example 3 using rabbit α-GNA1870 (TIGR-741) antibodies comparing 10 μl and 100 μl assay formats. As shown in FIG. 5, the 100 μl assays correlated with the results of SBA for two strains. Three additional strains tested showed positive results on SBA but negative for the WCE. The lower sensitivity observed with the WCE possibly due to the fixing process is likely the source of the issue. Antigen solubilized with detergent containing diluent is expected to correlate with the SBA results.

### Example 6

### Analysis of Results

This example illustrates and compares two methods of calculating a result from the ELISA assays. One method involves a simple calculation of the signal to noise ratio between the measured signal in the presence of the pathogen sample and the control with everything but the pathogen sample:

S/N=(OD_{bact}−OD_{subst})/(OD_{broth}−OD_{subst})

Wherein OD_{bact} is the measurement of the pathogen sample, OD_{subst} is the measurement of the control with substrate, and OD_{broth} is the measurement of the control with the broth in which the pathogen.

The second method involves calculating a percentage of maximum signal comparing the measured signal in the presence of the pathogen sample with a positive control with a pathogen known to be inhibited:

% Bₘₐₓ=100*(OD_{bact}−OD_{subst})/(OD_{positive control}−OD_{subst})

Wherein OD_{bact} is the measurement of the pathogen sample, OD_{subst} is the measurement of the control with substrate, and OD_{positive control} is the measurement of a positive control pathogen known to respond (e.g., H44/76 for GNA1870 or 5/99 for NadA). Advantages of the second method are that this method controls for growth of the bacteria, measurement of OD of bacteria in the broth, preparation of the sample and blanking of the plate, each of which could otherwise create differences between measurements made at different labs. Table 2 provides a comparison of results obtained by two different labs calculated using the second method for NadA.

TABLE 2
Comparison of second method results at
two different laboratories for NadA

| Strain | Lab 1 data | Lab 2 data |
|---|---|---|
| 5/99 | 100 | 100 |
| NMB | 87 | 104 |
| GB364 | 64 | 96 |
| 2996 | 47 | 56 |
| M4458 | 46 | 100 |
| F6124 | 6 | n.d. |
| MC58 | 3 | 4 |
| NZ98/254 | 2 | 0 |
| M3812 | 2 | 1 |
| H44/76 | 1 | 0 |

Table 3 provides a comparison of results obtained by two different labs calculated using the second method for GNA1870.

TABLE 3
Comparison of second method results at
two different laboratories forGNA1870

| | GNA1870 variant | | | |
|---|---|---|---|---|
| Strain | (expression level) | | Lab 1 data | Lab 2 data |
| H44/76 | 1.1 | (+++) | 100 | 100 |
| MC58 | 1.1 | (+++) | 95 | 109 |
| F6124 | 1.5 | | 16 | n.d. |
| NZ98/254 | 1.10 | (++) | 9 | 11 |
| M3812 | 1.9-2 | (++) | 7 | 10 |
| NMB | n.d. | (−) | 2 | 0 |
| 2996 | 2.1 | (+) | 2 | 0 |
| 5/99 | 2.8 | (−) | 2 | 1 |
| GB364 | 3.4 | (−) | 1 | 1 |
| M4458 | 2.10 | (n.d.) | 1 | 0 |
| M4407 | 2.4 | (−) | n.d. | 0 |
| 95N477 | 2.7 | (−) | n.d. | 0 |
| M1390 | 1.10 | (+++) | n.d. | 11 |
| GB013 | 2.4 | (++) | n.d. | 0 |

### Example 5

### Test of Monoclonal Antibodies

This example illustrates and compares use of monoclonal antibodies versus polyclonal antibodies used for detection (not capture) in the Ag ELISA method of Example 1. The ELISA assays were performed on various strains using either rabbit α-GNA1870 polyclonal antibodies or one of four different α-GNA1870 monoclonal antibodies MoAb Jar 1, MoAb Jar 5, MoAb Jar 10 and MoAb 502. An α-p24 monoclonal antibody was used as a negative control. Table 4 summarizes the results of the assays using the second method of Example 4.

TABLE 4
Comparison of monoclonal and polyclonal
antibodies for Ag ELISAs of GNA1870

| | poly- | | | | MAb | MAb |
|---|---|---|---|---|---|---|
| Strain (var) | clonal | Jar 1 | Jar 5 | Jar 10 | 502 | p24 |
| H44/76 (1.1) | 100 | 100 | 100 | 0 | 100 | 0 |
| MC58 (1.1) | 100 | 91 | 100 | 0 | 99 | 0 |
| 4243 (1.3) | 36 | 0 | 42 | 0 | 0 | 0 |
| M2937 (1.7) | 28 | 0 | 45 | 0 | 0 | 0 |
| F6214 (1.5) | 23 | 32 | 46 | 0 | 16 | 0 |
| M1390 (1.10) | 15 | 0 | 39 | 0 | 0 | 0 |
| UK101 (1.11) | 14 | 0 | 81 | 0 | 0 | 0 |
| NZ98/254 (1.10) | 13 | 0 | 33 | 0 | 0 | 0 |
| M3812 (1.9-2) | 12 | 5 | 19 | 0 | 42 | 0 |
| GB185 (1.9) | 8 | 0 | 8 | 0 | 0 | 0 |
| UK200 (1.9-3) | 5 | 2 | 3 | 0 | 17 | 0 |
| M0445 (1.8) | 4 | 1 | 5 | 0 | 14 | 0 |
| M6190 (1.6) | 4 | 0 | 0 | 0 | 0 | 0 |
| M4407 (2.4) | 1 | 0 | 0 | 0 | 0 | 0 |
| 5/99 (2.8) | 1 | 0 | 0 | 0 | 0 | 0 |

### Example 6

### Calibration of the Methods of Assessing Efficacy

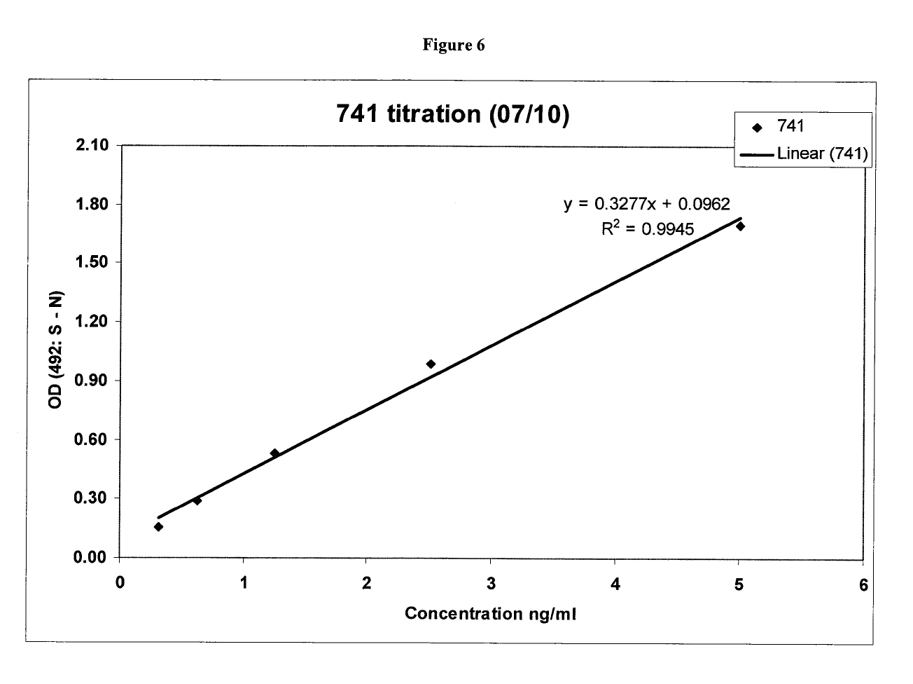

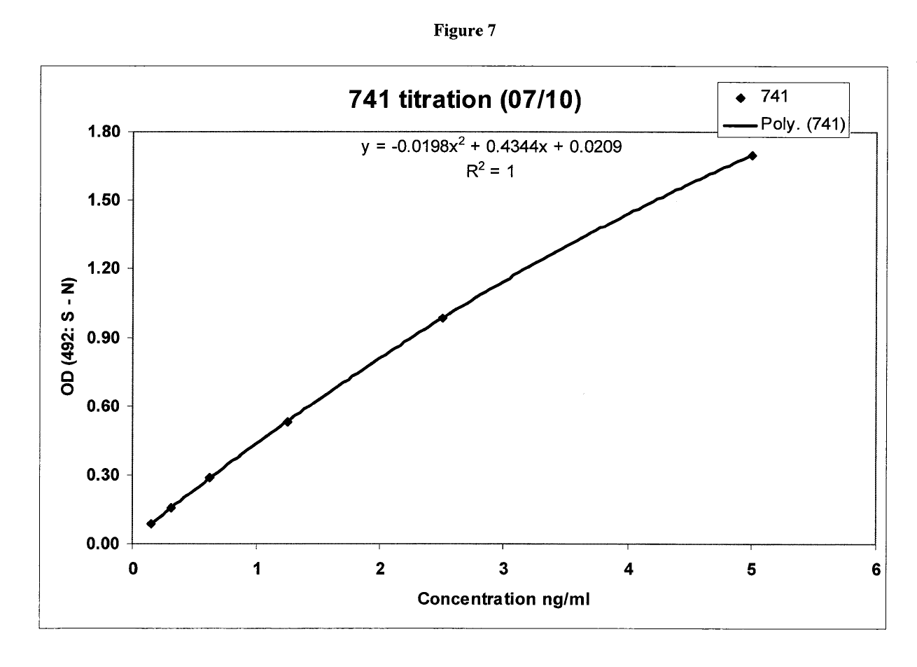

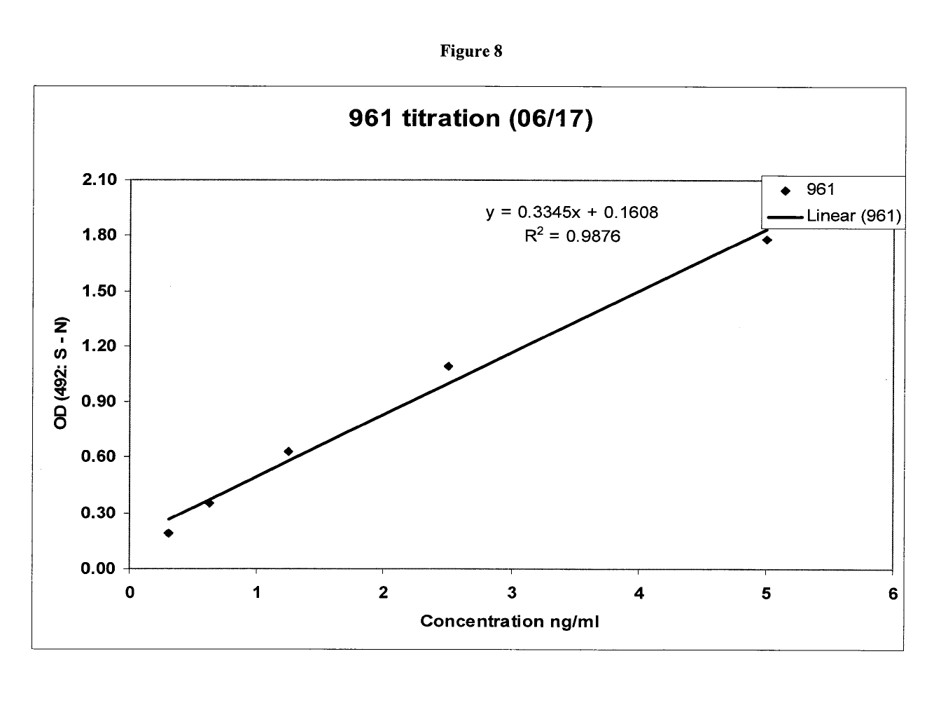

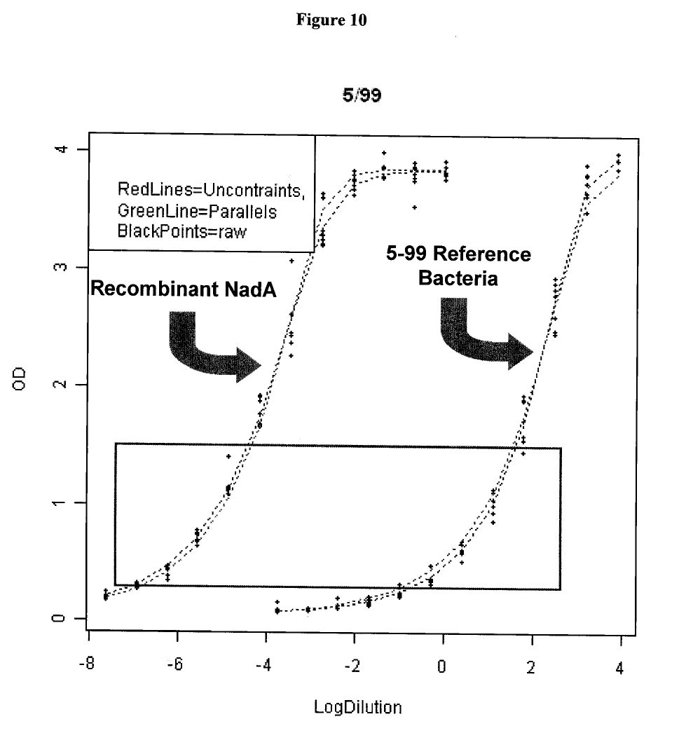

In order to use the methods disclosed herein in a quantitative manner, the measurements made with the test strain of interest need to be compared to a calibrated standard. Different methods of calibration of the measurements used to assess efficacy of vaccines were compared. The optical density at 492 nm (OD₄₉₂) was measured for five different concentrations of recombinant GNA1870 (TIGR-741) or recombinant NadA (TIGR-961). FIGS. 6 and 7 compare the linear fit and polynomial fit (five parameter logistic) of the five measurements of recombinant GNA1870 (TIGR-741). FIGS. 8 and 9 compare the linear fit and polynomial fit (five parameter logistic) of the five measurements of recombinant NadA (TIGR-961). From these figures, it is clear that a linear fit could work as long as the test bacteria is measured in the linear portion of the curve. However, a preferred method is to use the polynomial fit so that fewer dilutions of the test bacteria are required (i.e., the measurements of the bacteria do not necessarily need to be in the linear portion of the curve). Next, recombinant protein versus a reference bacterial strain were compared for utility in generating a calibration curve. FIG. 10 shows the OD₄₉₂ versus log dilution of recombinant NadA (TIGR-961) versus reference N. meningitidis strain 5/99. The box high-lights the divergence between the reference bacteria and the recombinant protein standard curves demonstrating that the reference bacteria are better as reference curve/calibrator.

With the polynomial fit to the reference bacterial standards, a method (referred to as the “meningococcal antigen typing system”, or MATS) was established for testing the efficacy of the three recombinant protein candidates designated GNA2132 (TIGR-287 or NHBA (“Neisserial Heparin Binding Antigen”)), GNA1870 (TIGR-741 or FHBP (the “factor H-Binding Protein”)), and NadA (GNA1994 or TIGR-961). The references standards used were, respectively, recombinant GNA2132-GNA, N. meningitidis strain Strain H44/76 and N. meningitidis strain 5/99. The MATs score (OD₄₉₂ measurement/path length) was generated for a set of test bacteria and the MATs scores were compared to the SBA (adult serum pools (post dose two with five component protein (including GNA2132, GNA1870, and NadA (5CVMB)+OMV (derived from N. meningitidis strain NZ98/254) vaccine (for details of the 5CVMB and OMV components, see Marzia M. Giuliani et al. PNAS (2006) 103:10834-10839)) for the test bacteria. The strains that were specific targets for each antigen (mismatching for all the other antigens) were selected. Those strains for each antigen were then rank ordered by MATS values. A range of MATs values between SBA positive and SBA negative were identified and the mid value was selected as the “positive bactericidal threshold” (PBT) for the MATs value for each antigen. Table 5 summarizes the PBT for each.

TABLE 5

| | Provisional PBT |
|---|---|
| GNA2132 | 0.9 |
| GNA1870 | 0.6 |
| NadA | 0.8 |

Using the provisional PBT, the accuracy of the MATs as a proxy for vaccine efficacy was compared to SBA (where SBA positive was deemed a true positive (i.e., efficacious response) for 84 N. meningitidis strains. The summary of the comparison of MATs to SBA is shown in Table 6.

TABLE 6

| | SBA positive | SBA Negative |
|---|---|---|
| MATS positive | 57 | 2 |
| MATS negative | 11 | 14 |
| Sensitivity | 0.84 | |
| Specificity | | 0.88 |

What we claim is:

1. A method of assessing efficacy of a vaccine component against a pathogen comprising the steps of: (a) providing a pathogen sample; (b) contacting the pathogen sample with a component binding antibody preparation; and (c) assessing efficacy of the vaccine component by detecting whether the component-directed antibody preparation binds to the pathogen sample.

2. The method of claim 1 wherein the pathogen sample is selected from the group consisting of an intact pathogen cell or virus and a detergent solubilized portion of the pathogen.

3. The method of claim 2 wherein the detergent is a non-ionic detergent, a cationic detergent, an anionic detergent, or a zwittergent.

4. The method of any of claims 1-3 wherein the detecting is performed with an ELISA assay.

5. The method of claim 4 wherein the enzyme of the ELISA assay is selected from horse-radish peroxidase, alkaline phosphatase, β-galactosidase, luciferase, and acetylcholinesterase.

6. The method of claims 4 or 5 wherein the ELISA assay uses a chromogenic, radiolabeled or a fluorescent substrate.

7. The method of any of claims 1-6 wherein the pathogen is selected from a bacterial pathogen, a viral pathogen, a fungal pathogen, a parasite pathogen, and a tumor.

8. The method of any of claims 1-6 wherein the pathogen is selected from N. meningitidis, N. gonorrhoeae, Streptococcus pyogenes, Streptococcus agalactiae, Streptococcus pneumoniae, H. influenzae, Staphylococcus aureus, Haemophilus influenza B, H. pylori, meningitis/sepsis associated E. coli, and uropathogenic E. coli.

9. The method of any of claims 1-6 wherein the pathogen is selected from influenza, RSV, HCV, HSV, HIV-1 and HIV-2.

10. The method of any of claims 1-9 wherein the vaccine component is a protein, a proteoglycan, a lipoprotein, a polysaccharide, a lipopolysaccharide, a viral envelope protein in monomeric or multimeric form, an outer membrane vesicle, a virus-like particle, or an entire vaccine.

11. The method of any of claim 1-10 wherein the antibody preparation is selected from a polyclonal antibody containing serum sample, polyclonal antibodies, antigen-purified polyclonal antibodies monoclonal antibodies, or a combination of two or more of the foregoing.

12. The method of claim 11 wherein the polyclonal antibodies are directed to the vaccine, to all components of the vaccine, or to a single component of the vaccine.

13. The method of claim 11 wherein the antibodies bind to the vaccine, to a single component of the vaccine or to an epitope of the vaccine.

14. The method of any of claims 1-13 wherein the pathogen is N. meningitidis serogroup B.

15. The method of claim 14 wherein the vaccine component comprises one or more of a GNA1870 antigen, a GNA2132 antigen, and a NadA antigen.

16. A method of assessing efficacy of a vaccine multicomponent N. meningitidis serogroup B against an N. meningitidis serogroup B strain comprising the steps of: (a) providing a detergent extracted sample of the N. meningitidis serogroup B strain; (b) separately contacting individual portions of the detergent extracted sample with a GNA1870 antigen-binding antibody preparation, a GNA2132 antigen-binding antibody preparation, and a NadA antigen-binding antibody preparation; and (c) assessing efficacy of the vaccine component by detecting whether each antibody preparation binds to contacted individual portion of the detergent extracted sample.

17. A kit for practicing any of the preceding claims.

18. The method of any of claims 1-16 further comprising the step of determining a positive bactericidal threshold by comparing the component-directed antibody preparation binding to a panel of reference pathogen samples with serum bactericidal assay results against the panel where the serum bactericidal assays are conducted using serum obtained from one or more subjects inoculated with the vaccine component.

19. The method of 18 wherein the assessing is performed by comparing the binding of each antibody preparation to the positive bactericidal threshold.
